(19) United States
(12) Patent Application Publication
(10) Pub. No.: US 2019/0317103 A1
(43) Pub. Date: Oct. 17, 2019

(54) GOLD-PLATINUM ALLOY NANOPARTICLES IN COLLOIDAL SOLUTIONS AND BIOLOGICAL APPLICATIONS USING THE SAME

(71) Applicant: IMRA America, Inc., Ann Arbor, MI (US)

(72) Inventors: Andrew A. Mills, Whitmore Lake (US); Kristin B. Cederquist, Vista, CA (US); Bing Liu, Ann Arbor, MI (US)

(21) Appl. No.: 16/314,499

(22) PCT Filed: Jul. 26, 2017
(86) PCT No.: PCT/US17/43989

§ 371 Date: Dec. 31, 2018

Related U.S. Application Data
(60) Provisional application No. 62/367,234, filed on Jul. 27, 2016.

(51) Int. Cl. G01N 33/58; G01N 33/53; C22C 5/04

(52) U.S. Cl. CPC G01N 33/587; G01N 33/53; B82Y 5/00; G01N 2469/10; G01N 2469/20; C22C 5/04

(57) ABSTRACT

Disclosed is a method of pulsed laser ablation production of gold-platinum AuₓPt₁₋ₓ alloy nanoparticles in a colloidal solution. The colloidal solution of AuₓPt₁₋ₓ alloy nanoparticles is suitable for many biological applications including lateral flow immunoassays and other bio-detections based on optical scattering. The nanoparticles form by fragmentation of the bulk material without evaporation, minimizing oxidation of the nanoparticles. The nanoparticles conjugate with bio-molecules such as protein, antibodies, peptides, RNA oligomers, DNA oligomers, other oligomers, or polymers effectively by passive adsorption. Advantageously the AuₓPt₁₋ₓ alloy nanoparticles have a wide optical extinction spectrum in the visible region, appearing nearly black in both colloidal and dried form. The nanoparticles can be used for labeling bio-molecules and provide a high visual contrast in visual-based bioassays. A combination of the near black color of the AuₓPt₁₋ₓ alloy nanoparticles with the red color of pure Au nanoparticles makes multiplexing bio-detection assays possible.

### RELATED APPLICATIONS

This application is a continuation of International Application No. PCT/US2017/043989, filed Jul. 26, 2017, which claims the benefit of which U.S. Provisional Application No. 62/367,234, filed Jul. 27, 2016, the entire disclosure of these applications being considered part of the disclosure of this application and hereby incorporated by reference.

### STATEMENT REGARDING FEDERALLY SPONSORED RESEARCH

NONE.

### TECHNICAL FIELD

This invention relates generally to nanoparticles for use in a variety of biological assays, and more specifically, to gold-platinum alloy nanoparticles for use in a variety of biological assays including in multiplexing assays.

### BACKGROUND OF THE INVENTION

Labeling of biological molecules, biomolecules, with small particles to generate signals for detection of the biological material is a method widely used in biochemical assays. In many assays a biomolecule is first labeled with a detectable signal particle to form a bio-conjugate and then this bio-conjugate is used to detect other bio-molecules. Alternatively, the small particles can be used to directly detect the presence of a biomolecule in a bio-conjugation reaction. The biochemical assays where these bio-conjugates are used include ELISA assays, lateral flow assays, Western blots, Northern blots, Southern blots, and other electrophoretic assays. Well-known examples of these small particles include colloidal solutions of gold nanoparticles which display a distinct red color caused by strong resonant plasmonic absorption near 530 nanometers (nm) of the gold nanoparticle. These gold nanoparticles can be used for optical and vision-based detection of biomolecules in a variety of assays. Other examples include magnetic nanoparticles which can be used in the magnetic detection of a variety of biomolecules either directly or through use of a bio-conjugate as described above.

Many biomolecules will bind with high affinity to the surface of pure gold nanoparticles by passive adsorption. Binding of biomolecules by passive adsorption to the surface of nanoparticles involves physically mixing the biomolecules with the nanoparticle colloid solution. The biomolecules will physically attach to the nanoparticle surface by the forces of electric attraction and hydrophobic interaction. Such composites of biomolecules with nanoparticles wherein the biomolecules are attached to the nanoparticle surface are also known as conjugates or bio-conjugates, and the process to produce such conjugates is known as bio-conjugation. Examples of these biomolecules that can be bound by passive adsorption include proteins, protein fragments, antibodies, peptides, RNA oligomers, DNA oligomers, other oligomers, and polymers. In addition, sometimes these biomolecules include functional groups, such as thiol groups, that also have affinity for the surface of gold nanoparticles and can contribute to the binding to the gold nanoparticles. Compared to covalent chemical conjugation methods, which are often inefficient and require complex and time consuming processes, passive adsorption simplifies the conjugation process and improves conjugation efficiency and surface loading of the nanoparticles. The capabilities of generating a strong optical signal and efficient binding with biomolecules make gold nanoparticles the primary choice to label biomolecules in many optical and visual-based bio-detection methods such as lateral flow immunoassays.

Nanomaterials that can provide color alternatives to the red of gold nanoparticles, but with the same surface and bio-conjugation properties are highly desired for multiplexing applications wherein one wants to detect more than one biomolecule on the same test strip. To detect more than one biomolecule it is necessary to have a color difference or some alternative detection method between the two biomolecules that are being detected. Incorporating dye molecules into particles comprising polymer or cellulose matrices is one example of a method of fabricating colorful particles; see for example Horii et al. JP2014163758A. These particles, however, require very different surface chemistry from gold nanoparticles and therefore will require alteration and optimization of protocols for use in biomolecule detection processes. They cannot be directly substituted in existing assays that utilize gold nanoparticles.

It is desirable to provide a method and system that will allow for detection of biomolecules that does not require a change in assay protocols and that can be used simultaneously with detection of biomolecules using gold nanoparticles.

### SUMMARY OF THE INVENTION

In general terms, this invention provides a method of fabrication of AuₓPt₁₋ₓ alloy nanoparticles that can be used for labeling biological molecules for biomedical diagnostic assays and other detection methods and the conjugation of biomolecules using the nanoparticles.

In an embodiment the present invention is a bio-conjugate comprising: an AuₓPt₁₋ₓ nanoparticle having a size range of from 10 nanometers (nm) to 100 nm and wherein x has a value of from 0.95 to 0.05, the AuₓPt₁₋ₓ nanoparticle having adsorbed thereon a plurality of bio-molecules and exhibiting an near black color. In some embodiments the value of x is more than 0.50, although less than 1.0 since these are alloys, and Au is the predominate component of the alloy nanoparticles.

In another embodiment the present invention is an assay for a bio-molecule comprising the steps of: a) providing a bio-conjugate comprising an AuₓPt₁₋ₓ nanoparticle having a size range of from 10 nm to 100 nm and wherein x has a value of from 0.95 to 0.05, said AuₓPt₁₋ₓ nanoparticle having adsorbed thereon a plurality of detector bio-molecules; b) exposing a sample comprising the bio-molecule to the bio-conjugate, wherein the detector bio-molecule binds to the bio-molecule while remaining adsorbed to the AuₓPt₁₋ₓ nanoparticle; and c) detecting the presence of the AuₓPt₁₋ₓ nanoparticle and thereby the presence of the bio-molecule in the sample.

These and other features and advantages of this invention will become more apparent to those skilled in the art from the detailed description of a preferred embodiment. The drawings that accompany the detailed description are described below.

### BRIEF DESCRIPTION OF THE DRAWINGS

FIG. 1 is a schematic illustration of a laser ablation system for utilization according to the present invention;

FIG. 2(a) is graph showing the size distribution of Au_{0.75}Pt_{0.25} nanoparticles produced according to the present invention;

FIG. 2(b) is a transmission electron microscope (TEM) image of Au_{0.75}Pt_{0.25} nanoparticles made according to the present invention;

FIG. 3(a) is an X-ray diffraction (XRD) pattern of an Au_{0.75}Pt_{0.25} sample according to the present invention and shows for comparison, the locations of pure Au and pure Pt peaks marked with thick solid lines and thick long dashed lines, respectively;

FIG. 3(b) shows expanded portions of the (111) diffraction peaks of an Au_{0.75}Pt_{0.25} sample, bottom curve, and an Au_{0.90}Pt_{0.10} sample, top curve, the samples are put on a Cu substrate during measurement so that the Cu (111) peaks can be used for alignment of the peak locations, the pure Au, pure Pt, and pure Cu (111) XRD peak locations are marked and labeled with solid lines, long dashed lines and short dashed lines respectively;

FIG. 3(c) shows the Ultra-violet-Visible (UV-Vis) absorption spectra of an Au_{0.75}Pt_{0.25} sample (solid line), an Au_{0.90}Pt_{0.10} sample (dashed line), and a pure Au sample (dotted line);

FIG. 3(d) is a photograph of a bottle of a colloidal Au_{0.75}Pt_{0.25} sample according to the present invention and shows a near black color;

FIG. 4 is a graph showing the adsorption capability of 40 micrograms of either pure Au nanoparticles 40 nm average diameter, circles, or Au_{0.75}Pt_{0.25} nanoparticles 50 nm average diameter, squares, for binding of Goat anti-mouse IgG antibody;

FIG. 5(a) is a photograph of two series of lateral flow immunochromatographic strips designed for detection of human chorionic gonadotropin (hCG) analyzed using either an Au-anti hCG bio-conjugate in the top series of strips or an Au_{0.75}Pt_{0.25}-anti-hCG bio-conjugate according to the present invention in the bottom series of strips as the signaling bio-conjugate, in both series, the lower line is the test line, whose intensity correlates with antigen level, which was varied from 1100 to 0 (mIU hCG/ml), and the upper line is the bio-conjugate intensity line;

FIG. 5(b) shows the quantitative colorimetric antigen detection capability of both the Au-anti hCG, shown in circles, and the Au_{0.75}Pt_{0.25}-anti hCG, shown in squares, bio-conjugates for the entire hCG antigen range evaluated;

FIG. 6(a) shows on the top portion a schematic representation of a multiplexed lateral flow assay in the presence of both hCG and human cardiac troponin I (cTnI) antigens as detected by Au_{0.75}Pt_{0.25}-anti hCG and Au-anti cTnI bio-conjugates, the bio-conjugates should capture the appropriate antigens, and then the bio-conjugates should be captured by the correct antibodies printed on the nitrocellulose strip, the bottom portion shows a photograph of an actual test run, 100 ng/ml hCG and 1000 ng/ml cTnI, showing both a line for the Au_{0.75}Pt_{0.25}-anti hCG and the Au-anti cTnI bio-conjugates; and

FIG. 6(b) shows an intensity quantification of anti-hCG and anti-cTnI immunochromatographic lines in the presence of both, either, or neither antigen at levels of 100 ng/ml hCG and 1000 ng/ml cTnI.

### DETAILED DESCRIPTION OF A PREFERRED EMBODIMENT

As discussed above, it is desirable to provide nanoparticles that can be used to form bio-conjugates that could be used in place of known gold nanoparticles and that would not require a change in assay procedures or conditions. In addition it would be helpful if these bio-conjugates had different colors from the standard gold nanoparticle color of red to allow for multiplex assays on the same test strip. Without significantly compromising the advantageous properties of gold nanoparticles, one way to adjust the nanoparticle color is to alloy gold with other metals. See for example Liu et al. U.S. Pat. Nos. 8,246,714 and 8,858,676 which teach methods of fabricating gold alloy nanoparticles by either high repetition rate short pulse duration pulsed laser ablation in liquid or burst laser ablation in a liquid, respectively. The advantages of these methods include the flexibility of adjusting alloy composition, defined as the atomic or weight ratio between the gold and the second metal, and the colloid purity. For example, by alloying Ag with different amounts of other atoms the color can be tuned between orange for an AgCu alloy with a high Ag content, green for an AgCu alloy with near equal Au and Cu content, and dark red for an AgCu alloy with a high Cu content. However, in the subsequent evaluations it was found that such alloy nanoparticles lack efficient passive binding to bio-molecules after storage. One reason appears to be that the nanoparticles gradually lose their negative surface charge due to adsorption of positive Ag⁺ or Cu⁺ ions dissolved in the water, and surface charge neutralization reduces affinity of the nanoparticles to proteins and destabilizes the colloids. For AuCu alloy nanoparticles, the color is gradually lost due to Cu oxidation, which changes the dielectric constant of the nanoparticles and red-shifts the plasmon resonance wavelength.

Darker colors for a bio-conjugate such as blue or black are also valuable in immunochromatographic detection, where the test strips are made with white nitrocellulose paper. Using a color other than red can provide better visual contrast, serve as a second color in multiplexing detections, and an alternative color can be necessary when the color of the assay sample, e.g., blood which is red, may complicate signal elucidation. The current invention introduces a method of fabricating AuₓPt₁₋ₓ nanoparticles that have a broad extinction spectrum with no distinct absorption peak in the visible region, providing a near black color.

The fabrication is based on pulsed laser ablation in water or other liquids of a bulk target material which is prefabricated and has the desired AuₓPt₁₋ₓ composition, wherein x is the atomic proportion of gold in the alloy and can be varied from 0.95 to 0.05. This means that a nanoparticle designated as Au_{0.90}Pt_{0.10} comprises 90 atomic % Au and 10 atomic % Pt. The laser beam is focused at or near the target surface by a focusing lens, and the ablated materials form nanoparticles in the liquid without any requirement for surfactant or stabilization agents. The particle size distribution is controlled by parameters such as laser power, focusing height, liquid electrical conductivity, ablation time, and pulse duration and frequency. This process results in a nanoparticle colloidal suspension in the ablation liquid that is very stable. The nanoparticle size distribution in the produced colloidal suspension can be further improved after ablation by means such as centrifugation or other methods of fractionation to recover the desired size range of nanoparticles.

After laser ablation, biomolecules including proteins, antibodies, protein fragments, peptides, oligomers, polymers, and biomolecules with a functional group such as thiol can be added to the colloidal suspension and bind to the nanoparticle surfaces by passive adsorption.

AuPt nanoparticles are known in the field of catalysis for their important catalytic properties. To maximize the surface to volume ratio, very small particle sizes below 5 nm are required for such applications, and the color of the nanoparticle is not particularly pertinent for such applications. For biological applications such as the present invention where optical properties are of major interest, much larger nanoparticle sizes on the order of greater than 20 nm are needed to enhance the optical scattering cross-section. However, AuPt nanoparticles of such size are not known to the biological field due to the lack of available materials in the desired size range.

AuPt nanoparticles are most often synthesized with chemical methods through co-reduction of constituent metal salts in a solvent. To control the size and composition, careful adjustment of reaction parameters such as the concentration of metal salts, the concentration of reducing agent and surfactant, the solvent temperature, and reaction time are necessary. An example of chemical synthesis of small AuPt nanoparticles to be used as the catalyst in fuel cells is disclosed in Zhong et al. U.S. Pat. No. 7,208,439. For larger sizes up to tens of nanometers, the reaction becomes harder to control in terms of composition and size distribution.

Pulsed laser ablation exhibits several advantages for metal alloy nanoparticle formation, including flexibility in adjusting alloy composition which can be accomplished by adjusting the bulk target composition. Direct formation in water eliminates the need for solvent transfer of the nanoparticles and results in high colloid suspension purity. In terms of types of lasers that have been used in the past for ablation, there are two main choices: a nanosecond pulsed laser with a pulse duration between 1-100 nanoseconds (ns), and an ultrashort pulsed laser with a pulse duration between about 1 femtosecond (fs) and 10 picoseconds (ps). The advantage of nanosecond pulsed laser is its high power and high ablation rates. The disadvantage is the high heat generated in the target material due to the accumulated heating during the long pulse, which leads to evaporation of the target materials.

For ablation in a liquid, the atomic species evaporated by the nanosecond laser ablation can react with the liquid and form oxides or hydroxides in water, or carbonates in organic liquids, all of which are undesired for the final nanoparticle products. The high heat may additionally decompose the liquid and bring in additional ligands to react with the target materials. For example, Nichols et al. (Nichols, W. T.; Sasaki, T. & Koshizaki, N.; Laser ablation of a platinum target in water. II. Ablation rate and nanoparticle size distributions, Journal of Applied Physics, 2006, vol 100, p114912) reports that the compound formed by Pt in water during nanosecond pulsed laser ablation, 1064 nm and 532 nm, is platinum hydroxide PtOH. On laser formation of AuPt alloys particles for catalysis applications, Zhang et al. (Jianming Zhang, Daniel Nii Oko, Sebastien Garbarino, Regis Imbeault, Mohamed Chaker, Ana C. Tavares, Daniel Guay, and Dongling Ma; Preparation of PtAu Alloy Colloids by Laser Ablation in Solution and Their Characterization; Journal of Physical Chemistry C, 2012, vol 116, pp 13413-13420) reported using an excimer laser with nanosecond pulse duration, wavelength 248 nm, and ablation in water. Zhang et al. demonstrated a variety of alloy compositions and small sizes of 5 nm or less. In that work, a wide range of pH values of from 4 to 11, preferring pH 11, were used to control the composition. This can be understood from the point of view of evaporation of Pt due to long pulse duration and subsequent quick oxidation by water. In comparison, the advantage of ultrashort pulsed laser ablation is its particular cold nature of material removal. This starts with a pulse duration of 1 fs to 10 ps, which is shorter than the time scale of excited hot electrons to reach equilibrium with the cold lattice. When the pulse is over, the material is left with a high temperature electron population and a cold lattice. Furthermore, for most materials heat conduction by electron diffusion and phonon-phonon interaction is slow or on the same order as electron-lattice interaction. Consequently the material temperature and stress rise faster than relaxation by heat dissipation and strain response. As a result, the material removal occurs by explosive mechanical disintegration of the bulk instead of slow evaporation as in nanosecond laser ablation, producing far fewer atomic species such as neutral atoms, ions, and small clusters of a few atoms, thus minimizing the chance of reaction with the liquid which generates undesired oxides or hydroxides impurities. Nanoparticles produced in this way conserve the bulk properties such as alloy compositions and compound stoichiometry.

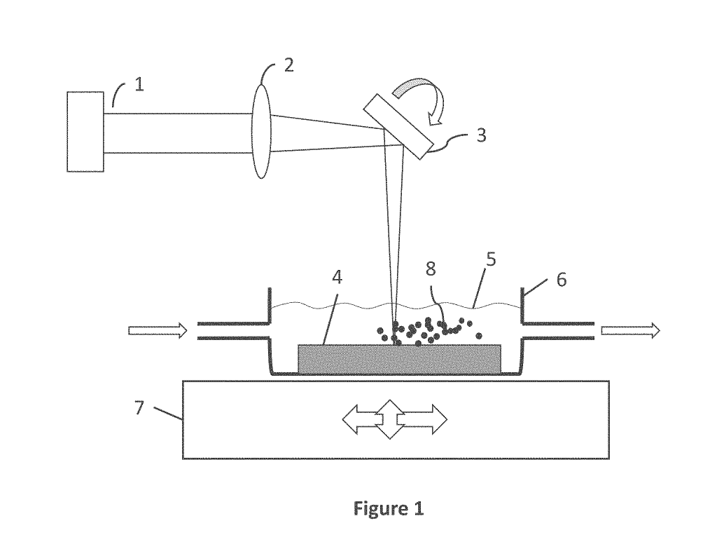

FIG. 1 illustrates an example of a laser ablation system for producing nanoparticles in a liquid according to the present invention. The laser beam 1 is focused by a lens 2 and guided by a vibrational mirror 3 onto the target 4. The target 4 is submerged in a liquid 5 contained in a container 6, which is positioned on top of a xyz motion stage 7. The nanoparticles 8 can be collected after laser ablation or during ablation in a flow system that passes the liquid 5 through the container 6.

For biological applications, water is the primary liquid chosen. Deionized water of high electrical resistance on the order of 10 MΩ·cm or higher is preferred. It has been determined that electrical conductivity is critical in controlling the particle size produced by ablation. To adjust the electrical conductivity, simple salts such as sodium chloride can be added to the liquid 5 before ablation to produce a low concentration of 10-100 microMolar (uM) of the salt to stabilize the electrical conductivity. Preferably the electrical conductivity is in the range of from about 1 to about 10 microS/cm. Preferably the pH of the liquid is neutral. There is no pH buffer required to adjust the pH or stabilizer to stabilize the colloid suspension. As will be shown in the biological application examples disclosed here in, it is an important advantage that the colloidal nanoparticle suspension is produced at neutral pH. If an acidic or basic pH is needed for producing the colloidal suspension, it will be in conflict with biological applications where bio-molecules have their own preferred pH for correct electric charge and functioning.

The preferred laser according to the present invention is an ultrafast pulsed laser with a pulse duration between about 1 fs to about 100 ps. In some embodiments the pulse duration is from about 1 fs to less than about 10 ps. The wavelength can be about 1 um or its second or third harmonics in the visible or UV region, respectively. The preferred pulse repetition rate is between about 0.1 to 1 MHz. A lower repetition rate can also be used as long as the production rate is acceptable. The preferred individual pulse energy is between about 1 microJoule (μJ) to 1 milliJoule (mJ), and may be in the range from about 10 to about 100 μJ. With an average power of a typical 10 Watts at 1 MHz, a 10 μJ pulse energy is appropriate. One advantage of a high repetition rate is that the plume of ablated material in an ionized plasma state may be subjected to multiple laser pulses before moving out of the laser focus, which helps to break down large particles and improve the nanoparticle size distribution. The disadvantage is accumulation of heat on the target surface, an imposition which can be mitigated by increasing the laser beam scanning speed.

The target 4 comprises an alloy of AuₓPt₁₋ₓ with composition x variable ranging from 0.95 to 0.05. Since the target is an alloy, meaning it has both Au and Pt in the composition, the nanoparticles produced according to the present invention always include both Au and Pt. In some embodiments preferably the alloy comprises predominantly Au, meaning x is greater than 0.50 and less than 1.00. In another embodiment x is 0.75 or greater. Such a target can be made by co-melting gold and platinum to form a uniform melt solution and then cooling the melt. Then X-ray diffraction (XRD) can be used to confirm the crystallinity and alloy composition of the target before ablation. Other methods such as energy dispersive x-ray (EDX) analysis are also convenient to check the alloy composition before use.

It is known that gold and platinum have a wide miscibility gap in bimetallic alloy compositions, meaning that in a solid alloy, gold and platinum segregate from each other and form local precipitates. The extent of segregation depends on the cooling rate of the melted alloy, and the slower the cooling rate the more segregation takes place. Because it is usually difficult to cool bulk metal melt very fast, a certain extent of segregation is unavoidable in the formed alloy target, which is a concern for fabrication of alloy nanoparticles. The miscibility gap is not an issue for those alloys with good affinity between the constituent metals such as Au—Ag systems and Au—Cu systems, where the miscibility gap is known to be zero. For immiscible metals, we have found that ultrashort pulsed laser ablation is very helpful as a remedy to improve the compositional uniformity of the produced nanoparticles. A reason for this is that when hot nanoparticles, which are always in liquid state right after ablation, are ejected into the ambient liquid during ablation, they are cooled down very rapidly due to the close contact with the liquid and the large surface to volume ratio of small nanoparticles. The cooling rate can be estimated to be 10³-10⁴ K/s, which is several orders of magnitude higher than a typical bulk cooling rate. Such a fast cooling rate can effectively prevent segregation and results in more uniform alloy nanoparticles.

Because of the small laser focal spot size, which can range from 0.05 to 1 millimeters, care must be taken to ensure that the extent of segregation in the bulk target is no larger than the laser focal spot. This can be monitored by EDX mapping, which has a sub-micron spatial resolution, of the bulk target 4.

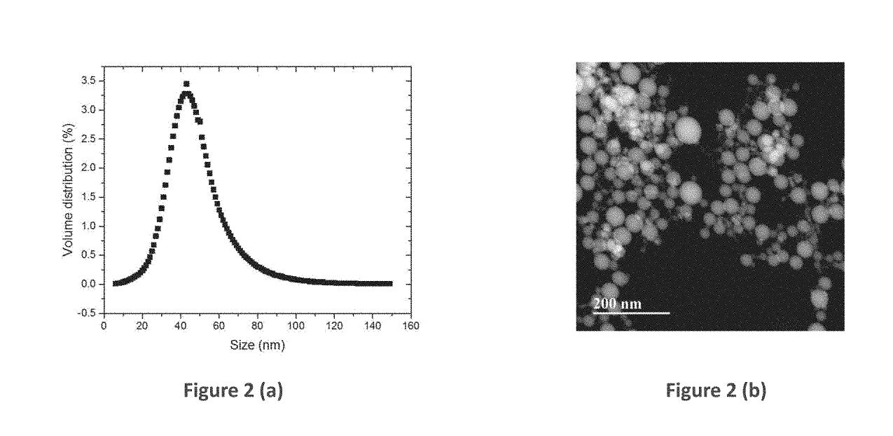

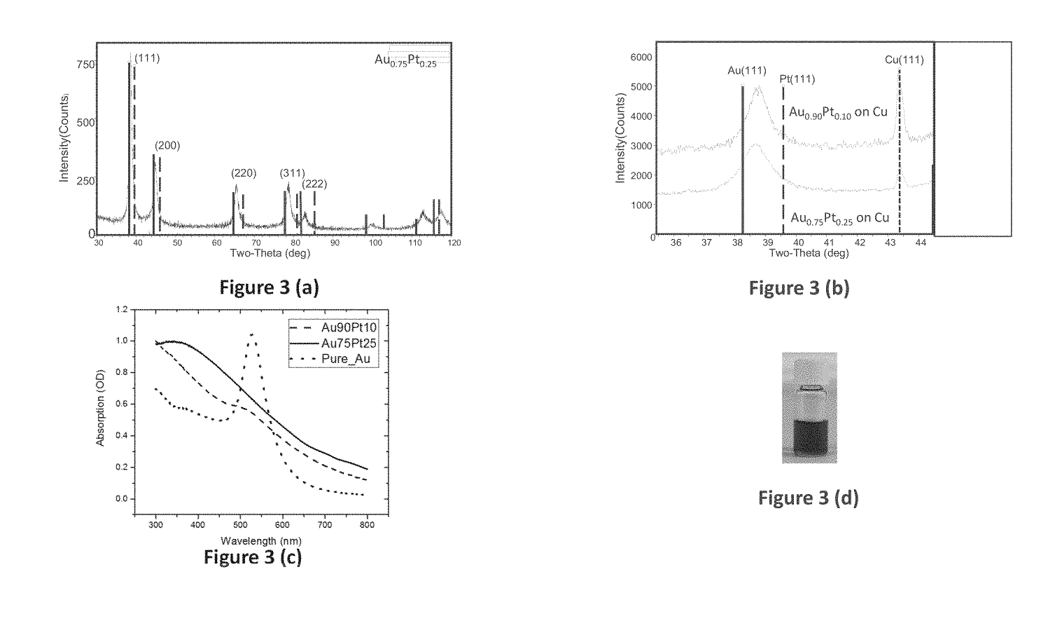

FIG. 2 (a) shows an example of the size distribution of a typical Au_{0.75}Pt_{0.25} alloy nanoparticle colloidal suspension made according to the present invention. The peak size is at 45 nm, which is ideal for the biological applications contemplated herein where optical scattering is the signal to be measured. In some embodiments the nanoparticles according to the present invention range from 40 to 60 nm. In the present invention the preferred particle size range of the AuₓPt₁₋ₓ nanoparticles according to the present invention range from about 10 to about 100 nm. It is well known that pure Au nanoparticles exhibit strong plasmonic scattering at an average particle diameter of 40 to 60 nm. For AuₓPt₁₋ₓ nanoparticles with x ranging from 0.95 to 0.05, as will be shown herein, see FIG. 3(c), there is no apparent plasmonic peak in the absorption (optical extinction) spectrum, therefore the scattering will mostly follow the Rayleigh law of scattering where the scattering intensity is proportional to the square of the particle volume, meaning the larger the particle, the stronger the scattering. Considering the low colloidal stability and difficulty in handling of large heavy particles a nanoparticle size range of 10 to 100 nm is most suitable for optical detection in biological applications. Further improvement of size distribution can be made by downstream size selection methods such as centrifugation or other means of fractionation. FIG. 2(b) is a dark field transmission electron microscope (TEM) image of the nanoparticles. Atomic absorption (AA) analysis was performed on the sample to determine the overall composition of the nanoparticles. The results measured the Pt atomic percent to be 28%, which is very close to the initial bulk target composition of 25 atomic % Pt.

FIG. 3 (a) shows a wide range of from 30° to 120°, theta-2theta mode XRD patterns of an Au_{0.75}Pt_{0.25} sample. Theoretical peak locations of pure Au and pure Pt are also marked with thick and long dash solid lines, respectively for comparison. It can be seen that the alloy particles have the same face-centered cubic (fcc) structure as the constituents. FIG. 3 (b) shows an expanded portion of the (111) peaks of an Au_{0.75}Pt_{0.25} sample and an Au_{0.90}Pt_{0.10} sample dried on copper substrates. Pure Au and pure Pt peaks are marked with thick solid and thick long dash lines for comparison. The Cu (111) peak is marked with a thick short dash line for alignment. Both samples exhibit broad XRD peaks due to the broadening effect of small nanoparticles. Theoretically there should be a shift towards the pure Au peak for the Au_{0.90}Pt_{0.10} sample due to its higher Au content compared with the Au_{0.75}Pt_{0.25} sample, but the shift is unclear in XRD due to the broadness of the peaks. Nevertheless it is apparent that both (111) peaks are located between the pure Au and pure Pt lines, supporting that the nanoparticles are made of alloys and have no segregation detectable by XRD. If there had been such segregation, each component would display a distinct set of peaks at the pure Au and pure Pt locations which would be discernable given the large separation between the pure Au and pure Pt XRD lines.

FIG. 3 (c) compares the UV-Vis absorption, more strictly known as optical extinction, spectra of an Au_{0.75}Pt_{0.25} sample, solid line, with an Au_{0.90}Pt_{0.10} colloid sample, dashed line, and a pure Au nanoparticle sample, dotted line. The pure Au nanoparticle sample has the well-known prominent plasmonic resonance absorption peak at 530 nm. By alloying with a small amount of Pt of 10 atomic percent the plasmonic peak is drastically reduced becoming a weak and broad bump near 520 nm superimposing on the background. When the Pt content is increased to 25 atomic percent in the Au_{0.75}Pt_{0.25} sample the spectrum exhibits broad and strong absorption from the UV to IR regions without any apparent plasmonic peaks and this spectrum remains the same after protein bio-conjugation to the nanoparticles. Such a broad spectrum produces a dark visual effect by the colloidal suspension, as illustrated in FIG. 3 (d) showing a picture of a bottle of an Au_{0.75}Pt_{0.25} sample which displays a dark brown color.

The colloidal suspension of nanoparticles produced according to the present invention is stable at room temperature, meaning 25° C., in the complete absence of stabilizing agents such as salts or surfactants. No aggregation or sedimentation of the nanoparticles is observed after storing at room temperature over 3 months. A long shelf lifetime of over half a year is seen. Similar stability is expected for bio-conjugates produced using the nanoparticles according to the present invention.

The high Au content of the nanoparticles is advantageous for antibody and protein conjugation by passive adsorption. For pure Au nanoparticles, protein and antibody passive adsorption is a low-cost and expedient bio-conjugation process widely used in immunoassay and bio-detection systems. For nanoparticles made of a new material, it is highly valuable to bioassay and bio-detection device manufactures for the bio-conjugation protocol to be unaltered from that of conventional pure Au nanoparticles.

Protein adsorption on the surface of colloidal metal nanoparticles is known to be a spontaneous process, with larger proteins exhibiting higher affinities for metal surfaces. This process is driven by a number of factors, including charge interaction (electrostatic attraction) and hydrophobic interaction, and is the primary means by which gold nanoparticles are bio-conjugated with proteins and antibodies for downstream electron microscopy staining or colorimetric immunochromatographic assays. Both pH and protein concentration are of critical importance in this reaction and must be optimized. A pH far away from the protein's isoelectric point will disrupt charge interactions, and insufficient protein addition will result in an unstable protein-nanoparticle conjugate, yielding aggregation upon salt or buffer exposure. Therefore in various embodiments, it can be is an important advantage that neutral pH is used during nanoparticle production, which allows freedom of optimizing the pH during biological applications such as protein adsorption.

After adsorption, nanoparticle blocking, centrifugation and washing to remove excess unreacted protein, adsorbed bio-conjugates can be displaced into solution through treatment with excess thiol (Mirkin et al., Anal. Chem. 2000, 72, pp 5535-5541). Labeling biomolecules with a fluorescent molecule before incubation with the nanoparticles provides a means by which the number of displaced proteins can be quantified. This experiment was employed to quantify the antibody binding capacity of Au_{0.75}Pt_{0.25} nanoparticles, in comparison to Au nanoparticles.

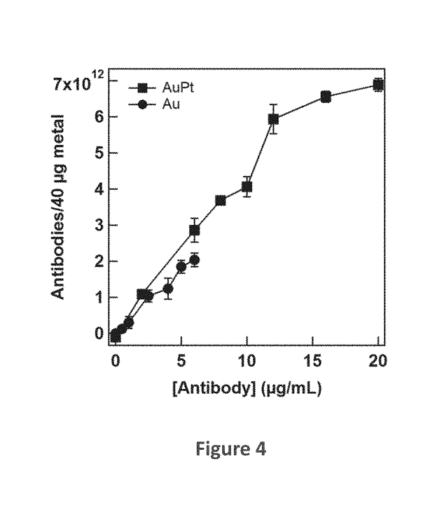

Fluorescently-labeled goat anti-mouse IgG was diluted to different concentrations and incubated with 40 μg of either 40 nm Au nanoparticles or 50 nm Au_{0.75}Pt_{0.25} nanoparticles at pH 7.4 for one hour at room temperature. FIG. 4 shows a comparison of the two antibody binding curves. It can be seen that at saturating conditions, the Au_{0.75}Pt_{0.25} nanoparticles were able to accommodate >3 times the number of antibodies per given weight of material as compared to the Au nanoparticles. This high adsorption capacity was completely unexpected and suggests that Au_{0.75}Pt_{0.25} nanoparticles have great potential as a label for colorimetric immunoassays. Little difference is seen in the binding capacity of nanoparticles in the size range of from 40 to 60 nm so the difference in binding capacity is not due to the small difference in size.

Biosensor performance has been shown to be highly correlated to the surface density of targeting biomolecules such as antibodies and DNA. This is especially true for lateral flow immunoassays, where the antibody immobilized on the nanoparticle must recognize its antigen, and this construct must be recognized by the antibody immobilized on the nitrocellulose membrane with only a limited amount of time, usually <15 minutes. In cases such as these, maximizing antibody loading per given amount of nanoparticle has been shown to increase assay sensitivity and widen dynamic range. The performance of Au_{0.75}Pt_{0.25} nanoparticles as a visual label in a lateral flow assay was evaluated by means of a sandwich assay to detect the pregnancy hormone human chorionic gonadotropin (hCG).

Monoclonal anti-hCG antibodies that were engineered for lateral flow immunoassays and hCG antigen were purchased from Scripps Laboratories and used without further purification. Lateral flow strips were fabricated by printing 0.5 μg of anti-hCG antibody as a test line and 0.5 μg of goat anti-mouse antibody, Lampire Biological Laboratories, per 0.5 cm-wide strip. Both Au and Au_{0.75}Pt_{0.25} nanoparticles were reacted with 6 μg/ml or 20 μg/ml anti-hCG antibody, respectively, at pH 8.2. After one hour of exposure, these nanoparticles were blocked with a 10 mg/mL solution of bovine serum albumin (BSA) for 30 minutes. Antibody-conjugated nanoparticles were then washed by centrifugation and re-suspended at ˜400 μg/mL metal concentration.

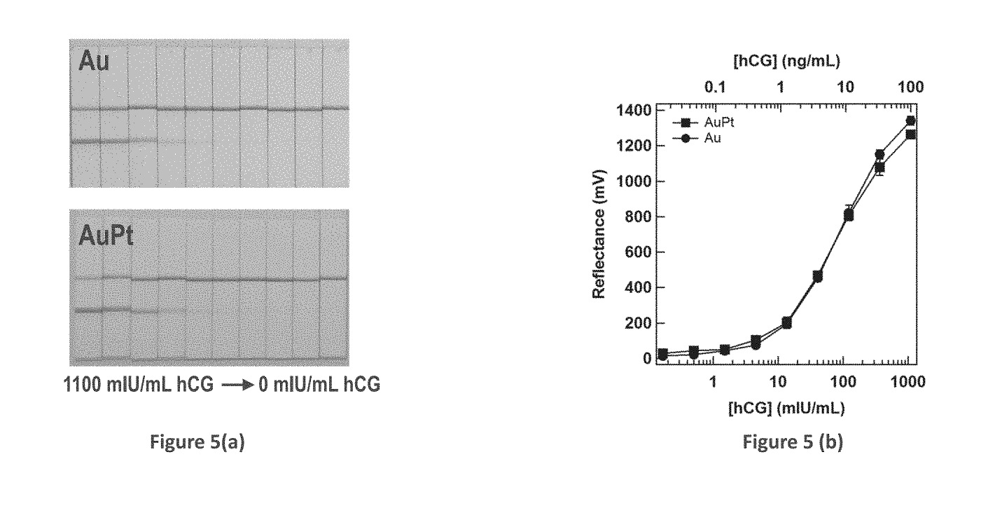

The hCG dilutions were made in running buffer, and 5 μL of nanoparticle conjugates were added to 50 μL of each dilution. Lateral flow strips were then dipped in the conjugate-antigen mixture and allowed to develop for 15 minutes. FIG. 5 (a) shows the photographs of these two conjugates employed in lateral flow assays with decreasing amounts of hCG antigen. All strips display a visible control line, thereby validating the results. Furthermore, while the Au nanoparticle conjugate strips showed their canonical red color, the Au_{0.75}Pt_{0.25} nanoparticle conjugate strips exhibited a striking black color, demonstrating that colloidal nanoparticle color is retained on the test strip. Quantification of the test line signal as a function of hCG antigen concentration is shown in FIG. 5 (b). The Au_{0.75}Pt_{0.25} particles exhibit equal sensitivity and dynamic range performance to Au nanoparticles, making them excellent alternatives to Au nanoparticles in lateral flow assays when another color is desired.

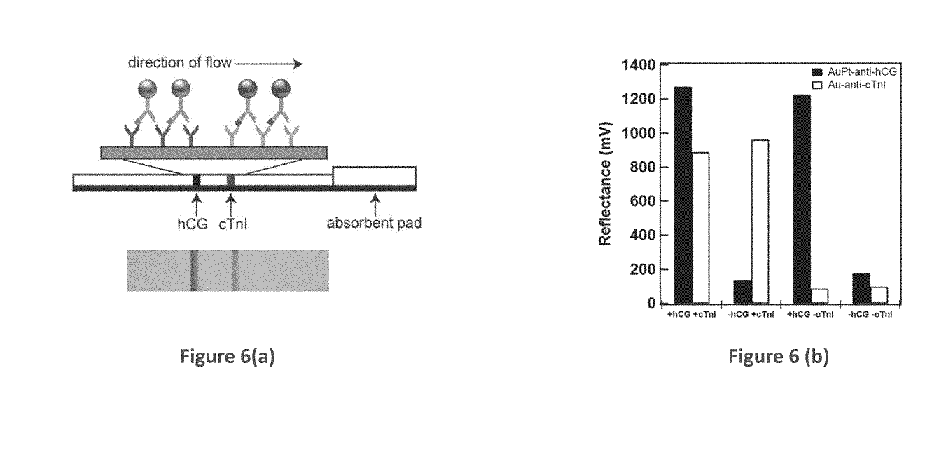

Multiplexed lateral flow assays with both Au and Au_{0.75}Pt_{0.25} nanoparticles can be accomplished by printing one line of antibodies against hCG and the other of antibodies against human cardiac troponin I (cTnI; HyTest) on the same test strip. Modification of the different nanoparticles with different targeting antibodies, Au_{0.75}Pt_{0.25}: anti-hCG and Au: anti-cTnI should result in the development of two different-colored lines when both antigens and particles are mixed together and allowed to run up the test strip. FIG. 6 (a) illustrates this concept both as a schematic and as a photograph, where both particles were mixed with 1000 ng/ml cTnI and 100 ng/ml of hCG before strip exposure. Clear differences in color can be elucidated, and the intensities of these lines are quantified in FIG. 6 (b). Signals were high upon the addition of both antigens; however, when an antigen was absent, the signal at that line dropped to a level coincident with the noise of the system for both lines evaluated. Small amounts of nonspecific binding occurred; however, these are likely antibody-dependent and can be minimized through assay optimization.

The AuₓPt₁₋ₓ nanoparticles according to the present invention will find broad use as a reporter label for a wide variety of bio-conjugates wherein a bio-molecule is conjugated to the AuₓPt₁₋ₓ nanoparticles according to the present invention. The bio-molecules contemplated include: proteins, peptides, antibodies, RNA oligomers, DNA oligomers, other oligomers, and polymers. The present AuₓPt₁₋ₓ nanoparticles also find use in multiplexing assays wherein Au nanoparticle labeled bio-conjugates are used along with AuₓPt₁₋ₓ nanoparticle labeled bio-conjugates and each are detectable as different colors on the same test strip.

In at least one embodiment the present invention includes a bio-conjugate comprising: a colloidal solution of AuₓPt₁₋ₓ nanoparticles in a liquid comprising water and having a particle size range of from 10 nm to 100 nm and wherein x has a value of from 0.95 to 0.05, the AuₓPt₁₋ₓ nanoparticles having adsorbed thereon a plurality of bio-molecules and exhibiting a near black color.

In at least one embodiment, the bio-molecule includes a protein, an antibody, a peptide, an oligomer, or a polymer.

In at least one embodiment, the bio-molecule is adsorbed onto the AuₓPt₁₋ₓ nanoparticle in an amount of up to 2×10¹² bio-molecules per microgram of AuₓPt₁₋ₓ nanoparticles.

In at least one embodiment, the colloidal solution of AuₓPt₁₋ₓ nanoparticles is stable at 25° C. for at least 3 months with no aggregation or sedimentation of the nanoparticles.

In at least one embodiment the present invention includes an assay for a bio-molecule comprising the steps of: a) providing a bio-conjugate comprising an AuₓPt₁₋ₓ nanoparticle having a particle size range of from 10 nm to 100 nm and wherein x has a value of from 0.95 to 0.05, the AuₓPt₁₋ₓ nanoparticle having adsorbed thereon a plurality of detector bio-molecules; b) exposing a sample comprising said bio-molecule to said bio-conjugate, wherein the detector bio-molecule binds to the bio-molecule while remaining adsorbed to the AuₓPt₁₋ₓ nanoparticle; and c) detecting the presence of the AuₓPt₁₋ₓ nanoparticle by measuring optical scattering and thereby detecting the presence of the bio-molecule in said sample.

In at least one embodiment, in the assay the bio-molecule includes an antigen and the detector bio-molecule is an antibody to the antigen.

In at least embodiment, in the assay the bio-molecule includes an RNA oligomer and the detector bio-molecule is a complementary RNA oligomer to the bio-molecule.

In at least one embodiment, in the assay the bio-molecule includes a DNA oligomer and the detector bio-molecule is a complementary DNA oligomer to the bio-molecule.

In at least one embodiment, the invention includes a colloidal solution of AuₓPt₁₋ₓ nanoparticles in a liquid comprising water, the AuₓPt₁₋ₓ nanoparticles having a particle size range of from 10 nm to 100 nm and wherein x has a value of from 0.95 to 0.05; and the AuₓPt₁₋ₓ nanoparticles exhibiting an X-ray diffraction pattern having a (111) peak located between the (111) peaks for pure Au and pure Pt.

The foregoing invention has been described in accordance with the relevant legal standards, thus the description is exemplary rather than limiting in nature. Variations and modifications to the disclosed embodiment may become apparent to those skilled in the art and do come within the scope of the invention. Accordingly, the scope of legal protection afforded this invention can only be determined by studying the following claims.

We claim:

1. A bio-conjugate comprising:
a colloidal solution of AuₓPt₁₋ₓ nanoparticles in a liquid comprising water and having a particle size range of from 10 nm to 100 nm and wherein x has a value of from 0.95 to 0.05, said AuₓPt₁₋ₓ nanoparticles having adsorbed thereon a plurality of bio-molecules and exhibiting a near black color.

2. A bio-conjugate as recited in claim 1, wherein said bio-molecule is a protein, an antibody, a peptide, an oligomer, or a polymer.

3. A bio-conjugate as recited in claim 1 wherein the bio-molecule is adsorbed onto the AuₓPt₁₋ₓ nanoparticle in an amount of up to 2×10¹² bio-molecules per microgram of AuₓPt₁₋ₓ nanoparticles.

4. A bio-conjugate as recited in claim 1 wherein said colloidal solution of AuₓPt₁₋ₓ nanoparticles is stable at 25° C. for at least 3 months with no aggregation or sedimentation of said nanoparticles.

5. An assay for a bio-molecule comprising the steps of:
a) providing a bio-conjugate comprising an AuₓPt₁₋ₓ nanoparticle having a particle size range of from 10 nm to 100 nm and wherein x has a value of from 0.95 to 0.05, said AuₓPt₁₋ₓ nanoparticle having adsorbed thereon a plurality of detector bio-molecules;
b) exposing a sample comprising said bio-molecule to said bio-conjugate, wherein said detector bio-molecule binds to said bio-molecule while remaining adsorbed to said AuₓPt₁₋ₓ nanoparticle; and
c) detecting the presence of said AuₓPt₁₋ₓ nanoparticle by measuring optical scattering and thereby detecting the presence of said bio-molecule in said sample.

6. The assay as recited in claim 5 wherein said bio-molecule is an antigen and said detector bio-molecule is an antibody to said antigen.

7. The assay as recited in claim 5 wherein said bio-molecule is an RNA oligomer and said detector bio-molecule is a complementary RNA oligomer to said bio-molecule.

8. The assay as recited in claim 5 wherein said bio-molecule is a DNA oligomer and said detector bio-molecule is a complementary DNA oligomer to said bio-molecule.

9. A colloidal solution of AuₓPt₁₋ₓ nanoparticles in a liquid comprising water, said AuₓPt₁₋ₓ nanoparticles having a particle size range of from 10 nm to 100 nm and wherein x has a value of from 0.95 to 0.05; and
said AuₓPt₁₋ₓ nanoparticles exhibiting an X-ray diffraction pattern having a (111) peak located between the (111) peaks for pure Au and pure Pt.
